(19) United States
(12) Patent Application Publication
(10) Pub. No.: US 2011/0178117 A1
(43) Pub. Date: Jul. 21, 2011

(54) OXAZOLOBENZIMIDAZOLE DERIVATIVES

(75) Inventors: Antonella Converso, Elkins Park, PA (US); Timothy John Hartingh, Blue Bell, PA (US); Sylwia Stachura, Lansdale, PA (US)

(73) Assignee: Merck Sharp & Dohme Corp., Rahway, NJ (US)

(21) Appl. No.: 13/120,930

(22) PCT Filed: Sep. 16, 2009
(86) PCT No.: PCT/US09/57099

§ 371 Date: Mar. 25, 2011

Related U.S. Application Data
(60) Provisional application No. 61/100,405, filed on Sep. 26, 2008.

(51) Int. Cl. A61K 31/437; C07D 498/04; A61K 31/424; C07D 401/14; A61K 31/454; C07D 498/14; A61P 25/18

(52) U.S. Cl. 514/293; 548/218; 514/375; 546/199; 514/322; 546/83

(57) ABSTRACT

The present invention is directed to oxazolobenzimidazole derivatives which are potentiators of metabotropic glutamate receptors, particularly the mGluR2 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### BACKGROUND OF THE INVENTION

The excitatory amino acid L-glutamate (sometimes referred to herein simply as glutamate) through its many receptors mediates most of the excitatory neurotransmission within the mammalian central nervous system (CNS). The excitatory amino acids, including glutamate, are of great physiological importance, playing a role in a variety of physiological processes, such as long-term potentiation (learning and memory), the development of synaptic plasticity, motor control, respiration, cardiovascular regulation, and sensory perception.

Glutamate acts via at least two distinct classes of receptors. One class is composed of the ionotropic glutamate (iGlu) receptors that act as ligand-gated ionic channels. Via activation of the iGlu receptors, glutamate is thought to regulate fast neuronal transmission within the synapse of two connecting neurons in the CNS. The second general type of receptor is the G-protein or second messenger-linked “metabotropic” glutamate (mGluR) receptor. Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McDonald and Johnson, Brain Research Reviews, 15, 41 (1990).

The present invention relates to potentiators of mGlu receptors, in particular mGluR2 receptors. The mGluR receptors belong to the Type III G-protein coupled receptor (GPCR) superfamily. This superfamily of GPCR's including the calcium-sensing receptors, GABAB receptors and pheromone receptors, which are unique in that they are activated by binding of effectors to the amino-terminus portion of the receptor protein. The mGlu receptors are thought to mediate glutamate's demonstrated ability to modulate intracellular signal transduction pathways. Ozawa, Kamiya and Tsuzuski, Prog. Neurobio., 54, 581 (1998). They have been demonstrated to be localized both pre- and post-synaptically where they can regulate neurotransmitter release, either glutamate or other neurotransmitters, or modify the post-synaptic response of neurotransmitters, respectively.

At present, there are eight distinct mGlu receptors that have been positively identified, cloned, and their sequences reported. These are further subdivided based on their amino acid sequence homology, their ability to effect certain signal transduction mechanisms, and their known pharmacological properties. Ozawa, Kamiya and Tsuzuski, Prog. Neurobio., 54, 581 (1998). For instance, the Group I mGluR receptors, which include the mGlu1R and mGIu5R, are known to activate phospholipase C (PLC) via Gαg-proteins thereby resulting in the increased hydrolysis of phosphoinositides and intracellular calcium mobilization. There are several compounds that are reported to activate the Group I mGlu receptors including DHPG, (R/S)-3,5-dihydroxyphenylglycine. Schoepp, Goldworthy, Johnson, Salhoff and Baker, J. Neurochem., 63, 769 (1994); Ito, et al., keurorep., 3, 1013 (1992). The Group II mGlu receptors consist of the two distinct receptors, mGluR2 and mGluR3 receptors. Both have been found to be negatively coupled to adenylate cyclase via activation of Gαi-protein. These receptors can be activated by a selective compound such as 1S,2S,5R,6S-2 aminobicyclo[3.1.0]hexane-2,6-dicarboxylate. Monn, et al., J. Med. Chem., 40, 528 (1997); Schoepp, et al., Neuropharmacol., 36, 1 (1997). This activitation leads to inhibition of glutamate release in the synapse (Cartmell et al. J Neurochem 75, 889 (2000)). Similarly, the Group III mGlu receptors, including mGluR4, mGluR6, mGluR7 and mGluR8, are negatively coupled to adenylate cyclase via Gαi and are potently activated by L-AP4 (L-(+)-2-amino-4-phosphonobutyric acid). Schoepp, Neurochem. Int., 24, 439 (1994).

Nonselective mGluR2/mGluR3 receptor agonists (Monn, et al., S. Med. Chem., 43, 4893, (2000)) have shown efficacy in numerous animal models of anxiety and psychosis as well as human clinical trials in schizophrenia patients; Patil et al, Nature Medicine, 13, 1102 (2007). Recent reports indicate that mGluR2 but not the mGluR3 receptor mediates the actions of the dual mGluR2/mGluR3 agonist LY379268 in mouse models predictive of antipsychotic activity. Woolley et al, Psycopharmacology, 196, 431 (2008). Additionally, recent animal studies demonstrate that selective potentiation of the mGluR2 receptor has similar effects to such non-selective agonists (Galici et al, Journal of Pharmacology and Experimental Therapeutics, 315, 1181 (2005)) suggesting an alternative strategy concerning the discovery of selective, positive allosteric modulators (PAMs or allosteric potentiators) of mGluR2 (Johnson et al, J. Med. Chem. 46, 3189, (2003); Pinkerton et al., J. Med. Chem., 47, 4595 (2004). These potentiators act by enabling the receptor to produce an enhanced response to endogenous glutamate. Such allosteric potentiators do not bind at the glutamate binding site also known as the “orthosteric site”, and may benefit by binding to a site other than the highly conserved orthosteric site. A potential advantage to this approach includes the opportunity to have a distinct pharmacological profile by enhancing the activity of the endogenous ligand upon its binding to the orthosteric site. The pharmacological distinctions include the potential for pharmacological specificity between related receptor types that share the same endogenous ligand. In addition, positive allosteric modulators of mGluR2 have been shown to potentiate the response of mGluR2 agonists such as LY379268 (Johnson et. Al. Biochemical Soc. Trans. 32, 881 (2004) and this represents an alternative strategy for treatment using mGluR2 selective PAMs.

It has become increasingly clear that there is a link between modulation of excitatory amino acid receptors, including the glutamatergic system, through changes in glutamate release or alteration in postsynaptic receptor activation, and a variety of neurological and psychiatric disorders. e.g. Monaghan, Bridges and Cotman, Ann. Rev. Pharmacol. Toxicol., 29, 365-402 (1989); Schoepp and Sacann, Neurobio. Aging, 15, 261-263 (1994); Meldrum and Garthwaite, Tr. Pharmacol. Sci., 11, 379-387 (1990). The medical consequences of such glutamate dysfunction make the abatement of these neurological processes an important therapeutic goal.

### SUMMARY OF THE INVENTION

The present invention is directed to oxazolobenzimidazole derivatives which are potentiators of metabotropic glutamate receptors, particularly the mGluR2 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### DETAILED DESCRIPTION OF THE INVENTION

The invention encompasses a genus of compounds of Formula I

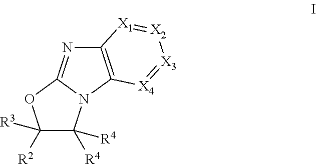

or a pharmaceutically acceptable salt thereof, wherein:
X₁, X₂, X₃ and X₄ are independently selected from the group consisting of: C(R¹) and N, wherein each R¹ is independently selected from the group consisting of:

(1) H,

(2) halo,

(3) C₁₋₈alkyl,

(4) C₂₋₆alkenyl,

(5) C₂₋₆alkynyl,

(6) C₃₋₆cycloalkyl,

(7) C₁₋₆alkoxy,

(8) C₃₋₆cycloalkoxy,

(9) —CN,

(10) —OH,

(11) —C(O)—O—C₁₋₄alkyl,

(12) —C(O)—C₁₋₄₄alkyl,

(13) —N(R)₂,

(14) —C(O)—N(R)₂,

(15) —S(O)ₖ—C₁₋₄alkyl, wherein k is 0, 1 or 2,

(16) -aryl,

(17) -heteroaryl, optionally substituted with 1 to 2 methyl groups,

(18) —C(O)-aryl,

(19) —N(R)-aryl,

(20) benzyl,

(21) benzyloxy,

(22) —CO₂H,

(23) —SH,

(24) —SO₂N(R)R,

(25) —N(R)C(O)N(R)R,

(26) —N(R)C(O)C₁₋₄alkyl,

(27) —N(R)SO₂N(R)R,

(28) trimethylsilyl and

(29) 1-methylsiletan-1-yl,

wherein groups (3) through (8) above are optionally substituted from one up to the maximum number of substitutable positions with one or more substituents independently selected from the group consisting of: OH, CN, oxo, halo, C₁₋₄alkoxy and C₁₋₄alkylamino, and two R¹ substituents on adjacent atoms may be joined together with the atoms to which they are attached to form a 5- or 6-membered saturated or partially unsaturated monocyclic ring optionally containing 1 or 2 heteroatoms selected from O, S and N, said ring optionally substituted with oxo or 1 to 3 halo groups, or both, and said ring optionally fused with a benzo group;

R² is selected from the group consisting of

(1)

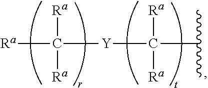

wherein Y is O or a bond, r and t are independently 0 to 9, except that r+t is greater than 4, and each R^{a} is independently selected from H, halo and C₁₋₄alkyl, optionally substituted with 1 to 3 halo groups, and two R^{a} groups on adjacent carbon atoms may be joined together to form a double bond,

(2) C₃₋₁₀cycloalkyl or C₃₋₁₀cycloalkyl —(CH₂)_{q}—, wherein q is 1 to 4,

(3) CF₃,

(4) tert-butyl,

(5) 2,2-dimethylpropyl and

(6)

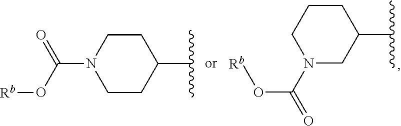

wherein R^{b} is selected from C₁₋₆alkyl, phenyl and benzyl, any of which may be optionally substituted with 1 to 3 halo groups;
R³ and each R⁴ are independently selected from the group consisting of: H, halo and C₁₋₄alkyl, said C₁₋₄alkyl optionally substituted with oxo and 1 to 3 substituents independently selected from the group consisting of: F, OH and N(R)₂; and
each R is independently selected from the group consisting of H and C₁₋₄alkyl.

Within the genus, the invention encompasses a first sub-genus of compounds of Formula I wherein R³ is methyl or ethyl.

Also within the genus, the invention encompasses a second sub-genus of compounds of Formula I wherein R² is

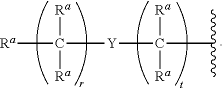

Also within the genus, the invention encompasses a third sub-genus of compounds of Formula I wherein R² is C₃₋₁₀cycloalkyl or C₃₋₁₀cycloalkyl —(CH₂)_{q}—, wherein q is 1 to 4.

Also within the genus, the invention encompasses a fourth sub-genus of compounds of Formula I wherein R² is

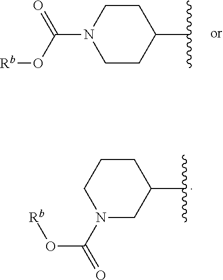

Also within the genus, the invention encompasses a fifth sub-genus of compounds having Formula Ia

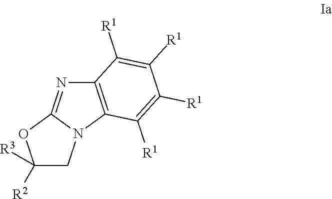

or a pharmaceutically acceptable salt thereof.

Within the fifth sub-genus, the invention encompasses a first class of compounds of Formula Ia wherein R² is

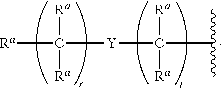

Also within the genus, the invention encompasses a sixth sub-genus of compounds having Formula Ib

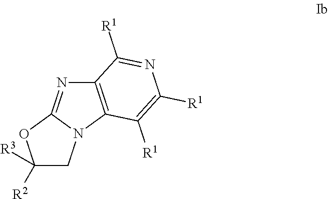

or a pharmaceutically acceptable salt thereof.

Within the sixth sub-genus, the invention encompasses a second class of compounds of Formula Ib wherein R² is

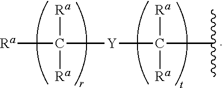

The invention also encompasses a compound selected from the following group:

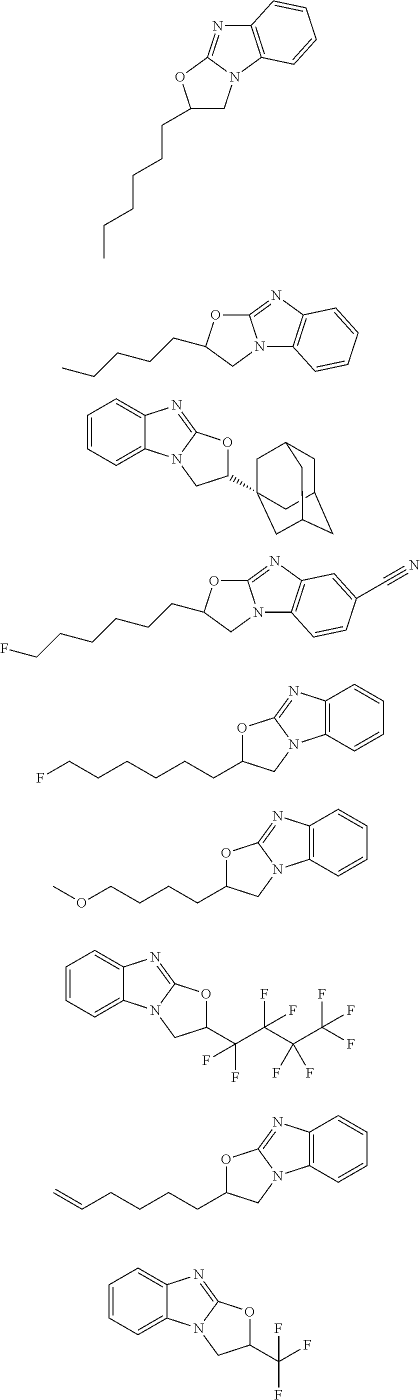

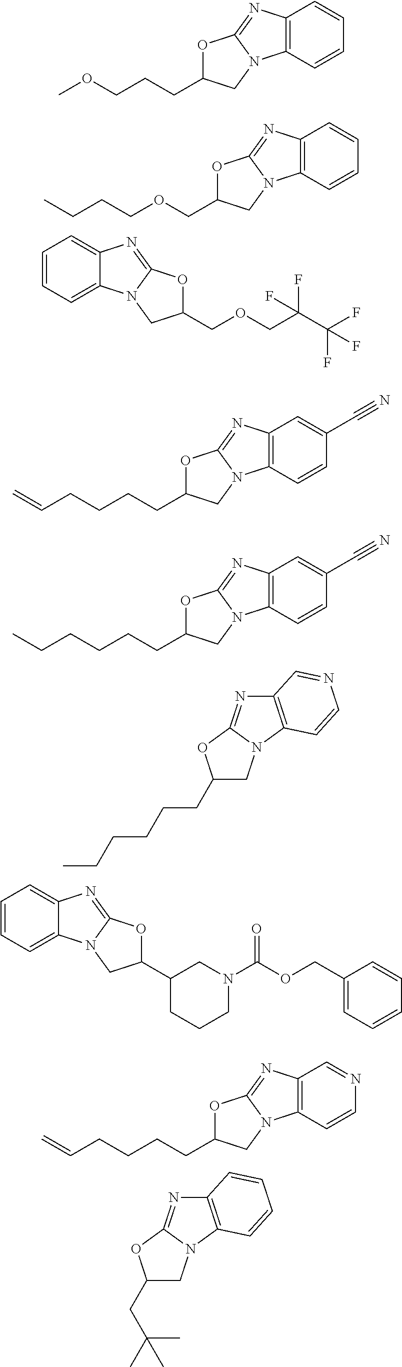

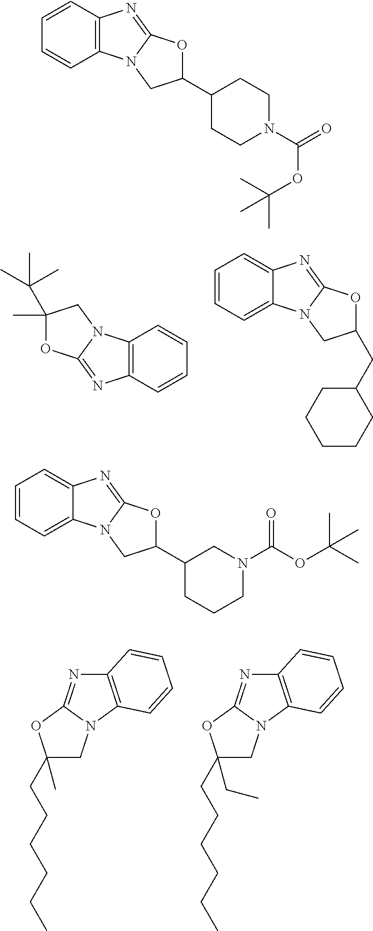

and pharmaceutically acceptable salts of the foregoing compounds.

The invention also encompasses a pharmaceutical composition comprising a compound of Formula Ia in combination with a pharmaceutically acceptable carrier.

The invention also encompasses a method for treating a neurological or psychiatric disorder associated with glutamate dysfunction in a patient in need thereof comprising administering to the patient a therapeutically effective amount of a compound of Formula I. The invention also encompasses this method wherein the neurological or psychiatric disorder associated with glutamate dysfunction is schizophrenia.

“Alkyl”, as well as other groups having the prefix “alk”, such as alkoxy, alkanoyl, means carbon chains which may be linear or branched or combinations thereof. Examples of alkyl groups include methyl, ethyl, propyl, isopropyl, butyl, sec- and tert-butyl, pentyl, hexyl, heptyl, octyl, nonyl, and the like.

“Alkylene” means a straight or branched chain of carbon atoms with a group substituted at both ends, such as —CH₂CH₂— and —CH₂CH₂CH₂—.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono-, bi- or tri-cyclic structures, optionally combined with linear or branched structures, having the indicated number of carbon atoms. Examples of cycloalkyl groups include cyclopropyl, cyclopentyl, cycloheptyl, adamantyl, cyclododecylmethyl, 2-ethyl-1-bicyclo[4.4.0]decyl, and the like.

“Alkoxy” means alkoxy groups of a straight or branched having the indicated number of carbon atoms. C₁₋₆alkoxy, for example, includes methoxy, ethoxy, propoxy, isopropoxy, and the like.

“Cycloalkoxy” means cycloalkyl as defined above bonded to an oxygen atom, such as cyclopropyloxy.

“Aryl” means mono- or bicyclic aromatic rings containing only carbon atoms. Examples of aryl include phenyl, naphthyl, indenyl, indenyl, tetrahydronaphthyl, 2,3-dihydrobenzofuranyl, dihydrobenzopyranyl, 1,4-benzodioxanyl, and the like.

“Heteroaryl” means mono- or bicyclic aromatic rings with at least one ring containing a heteroatom selected from N, O and S, and each ring containing 5 or 6 atoms. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

“Halogen” and “halo” includes fluorine, chlorine, bromine and iodine.

The compounds of the present invention are potentiators of metabotropic glutamate (mGluR) receptor function, in particular they are potentiators of mGluR2 receptors. That is, the compounds of the present invention do not appear to bind at the glutamate recognition site on the mGluR receptor, but in the presence of glutamate or a glutamate agonist, the compounds of the present invention increase mGluR receptor response. The present potentiators are expected to have their effect at mGluR receptors by virtue of their ability to increase the response of such receptors to glutamate or glutamate agonists, enhancing the function of the receptors. It is recognized that the compounds of the present invention would be expected to increase the effectiveness of glutamate and glutamate agonists of the mGluR2 receptor. Thus, the potentiators of the present invention are expected to be useful in the treatment of various neurological and psychiatric disorders associated with glutamate dysfunction described to be treated herein and others that can be treated by such potentiators as are appreciated by those skilled in the art.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. Any formulas, structures or names of compounds described in this specification that do not specify a particular stereochemistry are meant to encompass any and all existing isomers as described above and mixtures thereof in any proportion. When stereochemistry is specified, the invention is meant to encompass that particular isomer in pure form or as part of a mixture with other isomers in any proportion.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

The present invention also includes all pharmaceutically acceptable isotopic variations of a compound of the Formula I in which one or more atoms is replaced by atoms having the same atomic number, but an atomic mass or mass number different from the atomic mass or mass number usually found in nature.

Examples of isotopes suitable for inclusion in the compounds of the invention include isotopes of hydrogen such as 2H and 3H, carbon such as ¹¹C, ¹³C and ¹⁴C, nitrogen such as ¹³N and ¹⁵N, oxygen such as ¹⁵O, ¹⁷O and ¹⁸O, phosphorus such as ³²P, sulfur such as ³⁵S, fluorine such as ¹⁸F, iodine such as ²³I and ¹²⁵I, and chlorine such as ³⁶Cl.

Certain isotopically-labelled compounds of Formula I, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labelled compounds of Formula I can generally be prepared by conventional techniques known to those skilled in the art or by processes analogous to those described in the accompanying Examples using appropriate isotopically-labelled reagents in place of the non-labelled reagent previously employed.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids. It will be understood that, as used herein, references to the compounds of Formula I are meant to also include a pharmaceutically acceptable salts.

Exemplifying the invention are Examples 1-5, 1-10 to 1-19, 1-21, 1-23, 1-31, 2-5, 2-6, 3-3 to 3-7 and 4-2 to 4-4, described herein. The subject compounds are useful in a method of potentiating metabotorpic glutamate receptor activity in a patient such as a mammal in need of such inhibition comprising the administration of an effective amount of the compound. The present invention is directed to the use of the subject compounds disclosed herein as potentiators of metabotropic glutamate receptor activity. In addition to primates, especially humans, a variety of other mammals can be treated according to the method of the present invention.

The present invention is further directed to a method for the manufacture of a medicament for potentiating metabotropic glutamate receptor activity in humans and animals comprising combining a compound of the present invention with a pharmaceutical carrier or diluent.

The subject treated in the present methods is generally a mammal, preferably a human being, male or female, in whom potentiation of metabotropic glutamate receptor activity is desired. The term “therapeutically effective amount” means the amount of the subject compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with the disorders with an effective amount of the compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibitors of metabotropic glutamate receptor activity, in particular mGluR2 activity, may be demonstrated by methodology known in the art. Inhibition constants are determined as follows.

The compounds of the present invention may be tested in a fluorescence laser imaging plate reader (FLIPR) based assay. This assay is a common functional assay to monitor Ca²⁺ mobilization in whole cells expressing recombinant receptor coupled with a promiscuous G-protein. CHO dhfr-cells stably expressing recombinant human mGluR2 and Gα16 loaded with Fluo-4 AM (Invitrogen, Carlsbad Calif.) are treated with dose responses of compounds and the Ca²⁺ response is monitored on a FLIPR384 (Molecular Devices, Sunnydale Calif.) for agonist activity. The potentiation response is monitored after a subsequent addition of an EC20 concentration of glutamate (900 nM). The maximum calcium response at each concentration of compound for agonist or potentiation are plotted as dose responses and the curves are fitted with a four parameters logistic equation giving EC50 and Hill coefficient using the iterative non linear curve fitting software program.

The compounds of the present invention may also be tested in a [³⁵S]-GTPγS assay. The stimulation of [³⁵S]-GTPγS binding is a common functional assay to monitor Gαi-coupled receptor in native and recombinant receptor membrane preparation. Membrane from cells stably expressing hmGlu2 CHO-K1 (50 m) are incubated in a 96 well plate for 1 hour in the presence of GTPγS35 (0.05 nM), GDP (5 μM) and compounds. The reaction is stopped by rapid filtration over Unifilter GF/B plate (Packard, Bioscience, Meriden Conn.) using a 96-well cell harvester (Brandel Gaithersburg, Md.). The filter plates are counted using Topcount counter (Packard, Bioscience, Meriden Conn., USA). When compounds are evaluated as potentiators they are tested in the presence of glutamate (1 μM). The activation (agonist) or the potentiation of glutamate (potentiator) curves are fitted with a four parameters logistic equation giving EC₅₀ and Hill coefficient using the iterative non linear curve fitting software GraphPad (San Diego Calif., USA).

In particular, 1-5, 1-10 to 1-19, 1-21, 1-23, 1-31, 2-5, 2-6, 3-3 to 3-7 and 4-2 to 4-4 were tested and demonstrated activity in potentiating the mGluR2 receptor in the FLIPR assay, generally with an EC₅₀ of less than about 10 μM. Compounds within the present invention had activity in potentiating the mGluR2 receptor in the FLIPR and GTPγS assays with an EC₅₀ of less than about 1 μM. Examples 1-5, 1-10 to 1-19, 1-21, 1-23, 1-31, 2-5, 2-6, 3-3 to 3-7 and 4-2 to 4-4 resulted in a minimum 1.8-fold potentiation of glutamate response in the presence of an EC20 concentration of glutamate (900 nM). Such results are indicative of the intrinsic activity of the compounds in use as potentiators of mGluR2 receptor activity.

TABLE 1
Representative FLIPR EC₅₀ Values

| Ex. | EC50 | n | +/− (nM) |
|---|---|---|---|
| 1-5 | 21 nM | 4 | 15 |
| 1-19 | 11 nM | 2 | 2 |
| 1-21 | 3123 nM | 1 | N.A. |
| 1-31 | 68 nM | 2 | 11 |
| 2-7 | 935 nM | 1 | N.A. |
| 3-5 | 2139 nM | 1 | N.A. |
| 3-7 | 378 nM | 2 | 85 |
| 4-2 | 622 nM | 1 | N.A. |
| 4-3 | 2289 nM | 1 | N.A. |

Metabotropic glutamate receptors including the mGluR2 receptor have been implicated in a wide range of biological functions. This has suggested a potential role for these receptors in a variety of disease processes in humans or other species.

The compounds of the present invention have utility in treating, preventing, ameliorating, controlling or reducing the risk of a variety of neurological and psychiatric disorders associated with glutamate dysfunction, including one or more of the following conditions or diseases: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), autism, autism spectrum disorders, attention deficit/hyperactivity disorder, and conduct disorder.

Of the disorders above, the treatment of migraine, anxiety, schizophrenia, and epilepsy are of particular importance. In a preferred embodiment the present invention provides a method for treating migraine, comprising: administering to a patient in need thereof an effective amount of a compound of formula I. In another preferred embodiment the present invention provides a method for preventing or treating anxiety, comprising: administering to a patient in need thereof an effective amount of a compound of formula I. Particularly preferred anxiety disorders are generalized anxiety disorder, panic disorder, and obsessive compulsive disorder. In another preferred embodiment the present invention provides a method for treating schizophrenia, comprising: administering to a patient in need thereof an effective amount of a compound of formula I. In yet another preferred embodiment the present invention provides a method for treating epilepsy, comprising: administering to a patient in need thereof an effective amount of a compound of formula I.

Of the neurological and psychiatric disorders associated with glutamate dysfunction which are treated according to the present invention, the treatment of migraine, anxiety, schizophrenia, and epilepsy are particularly preferred. Particularly preferred anxiety disorders are generalized anxiety disorder, panic disorder, and obsessive compulsive disorder.

In an embodiment, the present invention provides a method for the treatment of schizophrenia comprising: administering to a patient in need thereof an effective amount of a compound of formula I or a pharmaceutical composition thereof. In one of the available sources of diagnostic tools, The Merck Manual (2006-2007), schizophrenia is characterized by psychosis (loss of contact with reality), hallucinations (false perceptions), delusions (false beliefs), disorganized speech and behavior, flattened affect (restricted range of emotions), cognitive deficits (impaired reasoning and problem solving), and occupational and social dysfunction. The skilled artisan will recognize that there are alternative nomenclatures, nosologies, and classification systems for neurological and psychiatric disorders, including migraine, and that these systems evolve with medical scientific progress

Thus, in an embodiment the present invention provides a method for treating migraine, comprising: administering to a patient in need thereof an effective amount of a compound of formula I or a pharmaceutical composition thereof. In one of the available sources of diagnostic tools, Dorland's Medical Dictionary (23'd Ed., 1982, W. B. Saunders Company, Philidelphia, Pa.), migraine is defined as a symptom complex of periodic headaches, usually temporal and unilateral, often with irritability, nausea, vomiting, constipation or diarrhea, and photophobia. As used herein the term “migraine” includes these periodic headaches, both temporal and unilateral, the associated irritability, nausea, vomiting, constipation or diarrhea, photophobia, and other associated symptoms. The skilled artisan will recognize that there are alternative nomenclatures, nosologies, and classification systems for neurological and psychiatric disorders, including migraine, and that these systems evolve with medical scientific progress.

In another embodiment the present invention provides a method for treating anxiety, comprising: administering to a patient in need thereof an effective amount of a compound of Formula I or a pharmaceutical composition thereof. At present, the fourth edition of the Diagnostic and Statistical Manual of Mental Disorders (DSM-IV) (1994, American Psychiatric Association, Washington, D.C.), provides a diagnostic tool including anxiety and related disorders. These include: panic disorder with or without agoraphobia, agoraphobia without history of panic disorder, specific phobia, social phobia, obsessive-compulsive disorder, post-traumatic stress disorder, acute stress disorder, generalized anxiety disorder, anxiety disorder due to a general medical condition, substance-induced anxiety disorder and anxiety disorder not otherwise specified. As used herein the term “anxiety” includes treatment of those anxiety disorders and related disorder as described in the DSM-IV. The skilled artisan will recognize that there are alternative nomenclatures, nosologies, and classification systems for neurological and psychiatric disorders, and particular anxiety, and that these systems evolve with medical scientific progress. Thus, the term “anxiety” is intended to include like disorders that are described in other diagnostic sources.

In another embodiment the present invention provides a method for treating depression, comprising: administering to a patient in need thereof an effective amount of a compound of Formula I or a pharmaceutical composition thereof. At present, the fourth edition of the Diagnostic and Statistical Manual of Mental Disorders (DSM-IV) (1994, American Psychiatric Association, Washington, D.C.), provides a diagnostic tool including depression and related disorders. Depressive disorders include, for example, single episodic or recurrent major depressive disorders, and dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, anxiety and phobias; seasonal affective disorder; or bipolar disorders or manic depression, for example, bipolar I disorder, bipolar II disorder and cyclothymic disorder. As used herein the term “depression” includes treatment of those depression disorders and related disorder as described in the DSM-IV.

In another embodiment the present invention provides a method for treating epilepsy, comprising: administering to a patient in need thereof an effective amount of a compound of Formula I or a pharmaceutical composition thereof. At present, there are several types and subtypes of seizures associated with epilepsy, including idiopathic, symptomatic, and cryptogenic. These epileptic seizures can be focal (partial) or generalized. They can also be simple or complex. Epilepsy is described in the art, such as Epilepsy: A comprehensive textbook. Ed. by Jerome Engel, Jr. and Timothy A. Pedley. (Lippincott-Raven, Philadelphia, 1997). At present, the International Classification of Diseases, Ninth Revision, (ICD-9) provides a diagnostic tool including epilepsy and related disorders. These include: generalized nonconvulsive epilepsy, generalized convulsive epilepsy, petit mal status epilepticus, grand mal status epilepticus, partial epilepsy with impairment of consciousness, partial epilepsy without impairment of consciousness, infantile spasms, epilepsy partialis continua, other forms of epilepsy, epilepsy, unspecified, NOS. As used herein the term “epilepsy” includes these all types and subtypes. The skilled artisan will recognize that there are alternative nomenclatures, nosologies, and classification systems for neurological and psychiatric disorders, including epilepsy, and that these systems evolve with medical scientific progress. The subject compounds are further useful in a method for the prevention, treatment, control, amelioration, or reduction of risk of the diseases, disorders and conditions noted herein.

The subject compounds are further useful in a method for the prevention, treatment, control, amelioration, or reduction of risk of the aforementioned diseases, disorders and conditions in combination with other agents, including an mGluR agonist.

The term “potentiated amount” refers to an amount of an mGluR agonist, that is, the dosage of agonist which is effective in treating the neurological and psychiatric disorders described herein when administered in combination with an effective amount of a compound of the present invention. A potentiated amount is expected to be less than the amount that is required to provided the same effect when the mGluR agonist is administered without an effective amount of a compound of the present invention.

A potentiated amount can be readily determined by the attending diagnostician, as one skilled in the art, by the use of conventional techniques and by observing results obtained under analogous circumstances. In determining a potentiated amount, the dose of an mGluR agonist to be administered in combination with a compound of formula I, a number of factors are considered by the attending diagnostician, including, but not limited to: the mGluR agonist selected to be administered, including its potency and selectivity; the compound of formula I to be coadministered; the species of mammal; its size, age, and general health; the specific disorder involved; the degree of involvement or the severity of the disorder; the response of the individual patient; the modes of administration; the bioavailability characteristics of the preparations administered; the dose regimens selected; the use of other concomitant medication; and other relevant circumstances.

A potentiated amount of an mGluR agonist to be administered in combination with an effective amount of a compound of formula I is expected to vary from about 0.1 milligram per kilogram of body weight per day (mg/kg/day) to about 100 mg/kg/day and is expected to be less than the amount that is required to provided the same effect when administered without an effective amount of a compound of formula I. Preferred amounts of a co-administered mGlu agonist are able to be determined by one skilled in the art.

The compounds of the present invention may be used in combination with one or more other drugs in the treatment, prevention, control, amelioration, or reduction of risk of diseases or conditions for which compounds of Formula I or the other drugs may have utility, where the combination of the drugs together are safer or more effective than either drug alone.

Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also includes therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

The above combinations include combinations of a compound of the present invention not only with one other active compound, but also with two or more other active compounds.

Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The pharmaceutical composition and method of the present invention may further comprise other therapeutically active compounds as noted herein which are usually applied in the treatment of the above mentioned pathological conditions.

In the treatment, prevention, control, amelioration, or reduction of risk of conditions which require potentiation of metabotorpic glutamate receptor activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

When treating, preventing, controlling, ameliorating, or reducing the risk of neurological and psychiatric disorders associated with glutamate dysfunction or other diseases for which compounds of the present invention are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligrams to about 50 milligrams. In the case of a 70 kg adult human, the total daily dose will generally be from about 7 milligrams to about 350 milligrams. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials are made according to procedures known in the art or as illustrated herein. The compounds of the present invention can be prepared in a variety of fashions.

### I. General Schemes

According to general scheme A, an epoxide (A-1) may be reacted with substituted 2-chloro or 2-bromobenzimidazoles (A-2) in the microwave and in the presence of cesium carbonate to provide, in one pot, the desired oxazolobenzimidazoles (A-4) via one pot cyclization of the intermediate alcohol (A-3).

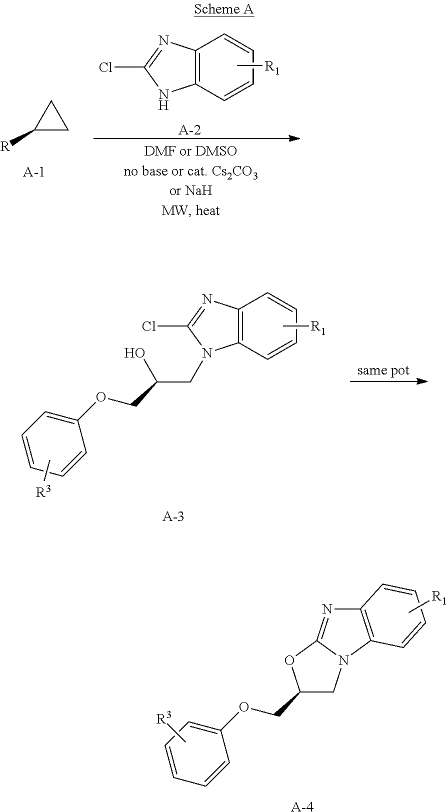

According to general scheme B, various heterocyclic derivatives can be synthesized. Substituted azabenzimidazolones (A-2) can be alkylated with substituted epoxides (B-1) under microwave conditions to provide a mixture of separable alkylated products (B-3 and B-4). Either of these products can be carried forward in an intramolecular Mitsunobu ring-closure to provide regioisomeric oxazolobenzimidazoles (B-5 and B-5′).

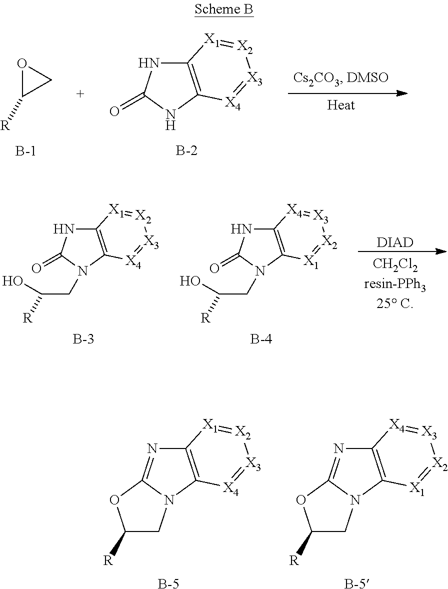

According to general scheme C, substituted 2-chloro or 2-bromobenzimidazoles (C-2) can be reacted with α-bromo- or α-chloroketone C-1 in the presence of base to provide ketone C-3. The ketone can then be reduced to the corresponding alcohol C-4 by treatment with sodium borohydride. Finally, alcohol C-4 can be cyclized under microwave irradiation to oxazolobenzimidazoles C-5 as a racemic mixture.

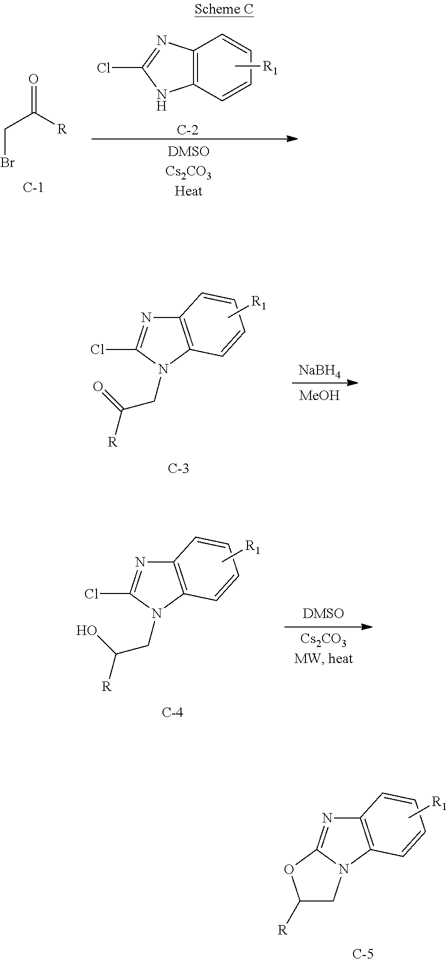

Oxazolobenzimidazoles substituted at the 2-positions (D-5) can be synthesized by a modification of the above procedure, whereby ketone D-3 is exposed to Grignard reagents to give tertiary alcohol D-4. Treatment of D-4 with base under microwave irradiation provides desired oxazolobenzimidazole D-5 as a racemic mixture.

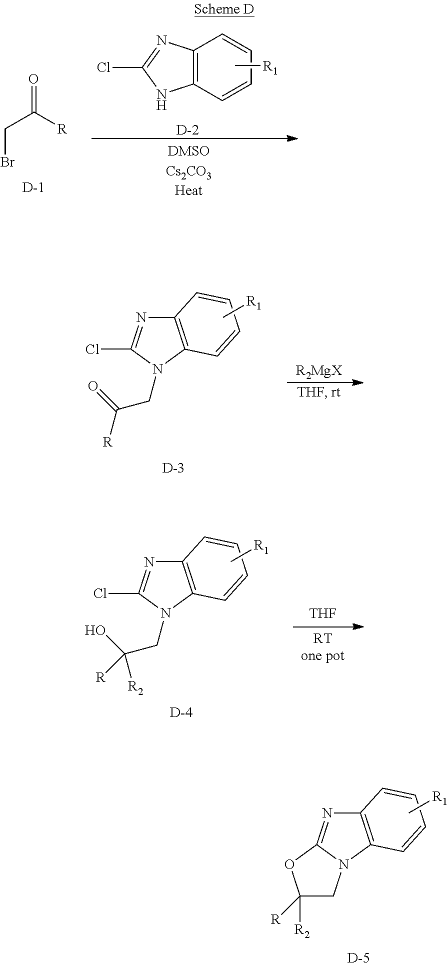

### II. Experimental Schemes

Certain reagents (phenols, epoxides and chlorobenzimidazoles) in the schemes below had to be synthesized prior to their incorporation in the inhibitor synthetic schemes. Specific procedures are described or referred to below:

### Synthesis of Chlorobenzimidazoles

Several chlorobenzimidazoles were not commercially available and had to be synthesized from their corresponding ortho-di-anilines as described for 2-chloro-5-cyanobenzimidazole in Ognyanov, V. I. et al J. Med. Chem. 2006, 49, 3719-3742.

### Synthesis of Epoxides (E Schemes)

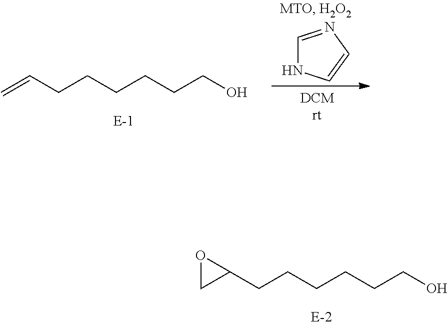

### 6-(oxiran-2-yl)hexan-1-ol (E-2)

To a solution of oct-7-en-1-ol (E-1) (2.24 g, 17.5 mmol, 1 eq) in DCM (9 mL) was added pyrazole (143 mg, 2.1 mmol, 0.12 equiv), MTO (22 mg, 0.09 mmol, 0.005 eq) and 35% aqueous hydrogen peroxide (3.4 mL, 34.9 mmol, 2 eq). The mixture was stirred at room temperature until disappearance of starting material as monitored by TLC, it was then cooled with an ice bath and a small amount of manganese dioxide was added. The organic layer was separated, the remaining aqueous phase was extracted with DCM and the combined organic phases were, dried over sodium sulfate and concentrated to yield 6-(oxiran-2-yl)hexan-1-ol (E-2) as a colorless oil, of sufficient purity for following manipulations. ¹H NMR (500 MHz, CDCl₃): δ 3.63 (t, J=6.6 Hz, 2H); 2.93-2.88 (m, 1H); 2.75 (t, J=4.5 Hz, 1H); 2.47 (dd, T=5.1, 2.8 Hz, 1H); 1.60-1.42 (m, 6H); 1.41-1.34 (m, 4H).

The compounds shown in Table 1 were synthesized according to Scheme E.

TABLE 1

| | | |
|---|---|---|
| E-3 | 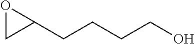 | 4-(oxiran-2- yl)butan-1-ol |
| E-4 | 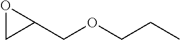 | 2-(propoxymethyl) oxirane |
| E-5 | 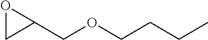 | 2-(butoxymethyl) oxirane |

Synthesis of a-bromoketones (K schemes)

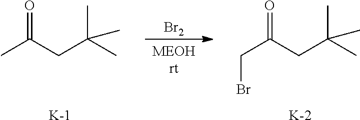

To a solution of 4,4-dimethylpentan-2-one (K-1) (150 mg, 1.314 mmol, 1 eq.) in MeOH (1 mL), bromine was added (68 μL, 1.314 mmol, 1 eq.) and the mixture was stirred at room temperature until disappearance of starting material as monitored by NMR. The solvent was then removed yielding desired bromoketone K-2 as a clear liquid, of sufficient purity for following manipulations.

The compounds shown in Table 2 were synthesized according to Scheme K.

TABLE 2

| | | |
|---|---|---|
| K-3 | 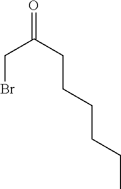 | 1-bromooctan- 2-one |
| K-4 | 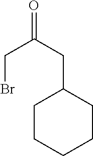 | 1-bromo-3- cyclo- hexylacetone |
| K-5 | 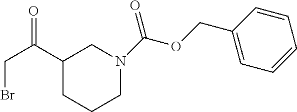 | Tert-butyl 3- (bromoacetyl) piperidine- 1-carboxylate |
| K-6 | 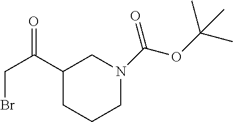 | Benzyl 3- (bromoacetyl) piperidine-1- carboxylate |

Synthesis of mGluR2 Inhibitors—Oxazolobenzimidazoles

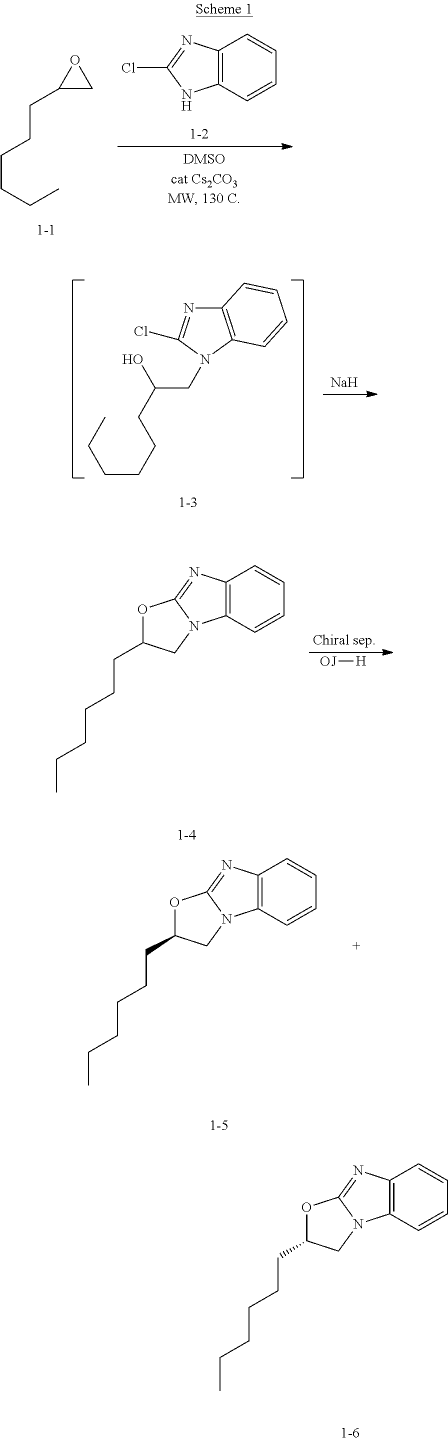

### Example 1-5

### (2R)-2-hexyl-2,3-dihydro[1,3]oxazolo[3,2-a]benzimidazole (1-5)

A mixture of 2-hexyloxirane (1-1) (277 mg, 2.16 mmol, 1 eq.), 2-chlorobenzimidazole (1-2) 330 mg, 2.16 mmol, 1.0 eq.) and cesium carbonate (35 mg, 0.1 mmol, 0.05 eq.) in DMSO (2 mL) was heated at 13° C. in a microwave until disappearance of SM as monitored by LC-MS. To the cooled mixture, containing both alcohol 1-3 and fully cyclized product 1-4, NaH (16 mg of 60% suspension in mineral oil, 0.32 mmol, 0.5 eq.) was added, followed by stirring at room temperature. Once evolution of hydrogen ceased, the mixture was again heated in the microwave at 130 C until full conversion to desired product as monitored by LC-MS. Water was then added and the solid precipitate was filtered and purified by silica gel chromatography to yield racemic 2-hexyl-2,3-dihydro[1,3]oxazolo[3,2-d]benzimidazole (14) as a white, crystalline solid. Chiral separation on an OJ-H column afforded (2R)-2-hexyl-2,3-dihydro[1,3]oxazolo[3,2-a]benzimidazole (1-5) and (25)-2-hexyl-2,3-dihydro[1,3]-oxazolo[3,2-a]benzimidazole (1-6). ¹H NMR (400 MHz, CD₃OD) δ 7.36 (m, 1H), 7.24 (m, 1H), 7.12 (m, 2H), 5.47 (m, 1H), 4.85 (s, 1H), 4.45 (dd, 1H, J=9.15, 8.15 Hz), 3.95 (dd, 1H, J=9.25, 7.33 Hz), 3.30 (dd, 1H, J=1.64, 1.56 Hz), 1.80-2.05 (m, 2H), 1.2-1.7 (m, 8H), 0.9 (t, 3H, J=5.1 Hz). LRMS m/z (M+H) 245.1 found, 245.2 required.

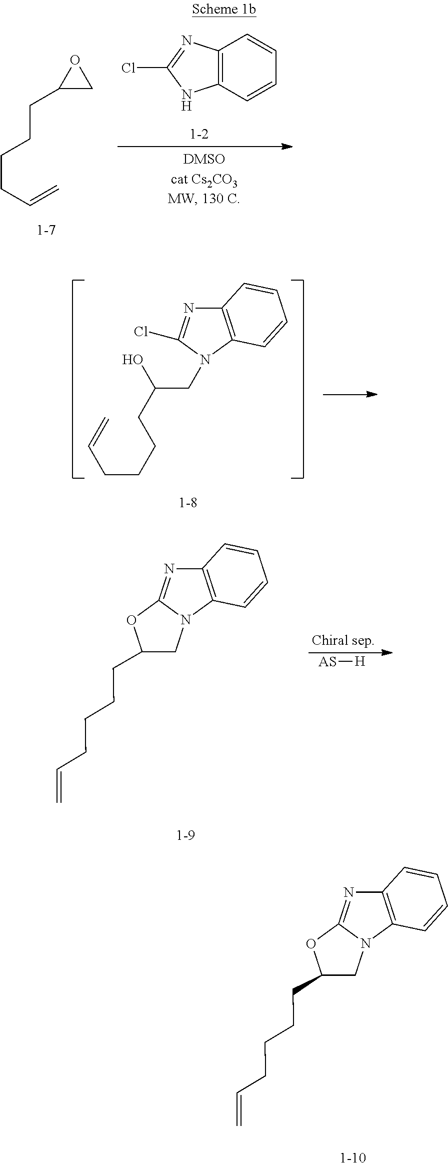

### Example 1-10

### (2R)-2-hex-5-en-1-yl-2,3-dihydro[1,3]oxazolo[3,2-a]benzimidazole (1-10)

A mixture of 2-hex-5-en-yloxirane (1-7) (500 mg, 3.96 mmol, 1.0 eq.), 2-chlorobenzimidazole (1-2) (623 mg, 4.08 mmol, 1.03 eq.) and cesium carbonate (1291 mg, 3.96 mmol, 1.0 eq.) in DMSO (4 mL) was heated at 130 C in a microwave until disappearance of SM as monitored by LC-MS. The mixture was cooled, the crude was filtered through a silica plug and the residue was purified by reverse phase liquid chromatography (Sunfire C18 OBD 5 μm, 20×150 mm column; 0-100% CH₃CN/H₂O gradient w/0.1% TFA present). The product in CH₃CN/H₂O was poured into aqueous sodium carbonate solution (2.0 M, 5 mL) and extracted with ethyl acetate. The organic layer was separated, dried over sodium sulfate and concentrated to yield 2-hex-5-en-1-yl-2,3-dihydro[1,3]oxazolo[3,2-a]benzimidazole (1-9) as a white, crystalline solid. Chiral separation on Chiralcel AS-H column (15% MeOH in hexanes with 0.1% DEA) afforded (2R)-2-hex-5-en-1-yl-2,3-dihydro[1,3]oxazolo[3,2-a]benzimidazole (1-10). ¹H NMR (500 MHz, CDCl₃): δ 7.53 (d, J=7.8 Hz, 1H); 7.19-7.13 (m, 1H); 7.11 (d, J=4.3 Hz, 2H); 5.80 (ddt, J=17.2, 10.2, 6.6 Hz, 1H); 5.40-5.33 (m, 1H); 5.06-4.93 (m, 2H); 4.34 (t, J=8.4 Hz, 1H); 3.87 (t, J=8.0 Hz, 1H); 2.10 (q, J=6.7 Hz, 2H); 2.08-1.99 (m, 1H); 1.92-1.83 (m, 1H); 1.64-1.53 (m, 2H); 1.50 (t, J=7.2 Hz, 4H). LRMS m/z (M+H) 243.0 found, 243.32 required.

The compounds shown in Table 3 were synthesized according to Scheme 1 b.

TABLE 3

| | | | |
|---|---|---|---|
| Ex. 1-11 | 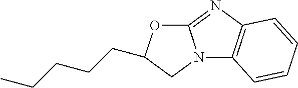 | 2-pentyl-2,3- dihydrobenzo[d]oxazolo [3,2-a]imidazole | HRMS m/z (M + H) 231.14 found, 231.14 required. |
| Ex. 1-12 | 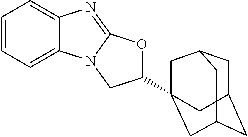 | (2R)-2-[(1S,3R,5R,7S)]- 1-adamantyl]-2,3- dihydro[1,3]oxazolo [3,2-a]benzimidazole | LRMS m/z (M + H) 243.1 found, 243.3 required. |
| Ex. 1-13 | 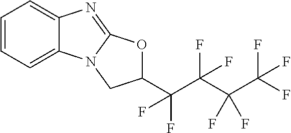 | 2-(nonafluorobutyl)-2,3- dihydro[1,3]oxazo1o [3,2-a]benzimidazole | LRMS m/z (M + H) 378.8 found, 378.1 required. |
| Ex. 1-14 | 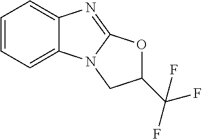 | 2-(tirfluoromethyl)-2.3- dihydro[1,3]oxazolo [3,2-a]benzimidazole | LRMS m/z (M + H) 229.0 found, 229.1 required. |
| Ex. 1-15 | 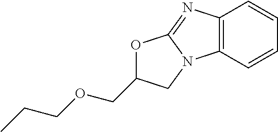 | 2-(propoxymethyl)-2,3- dihydrobenzo[d]oxazolo [3,2-a]imidazole | LRMS m/z (M + H) 229.0 found, 229.1 required. |
| Ex. 1.16 | 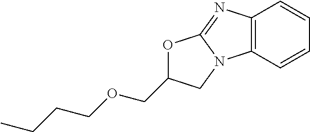 | 2-(butoxymethyl)-2,3- dihydrobenzo[d]oxazolo [3,2-a]imidazole | LRMS m/z (M + H) 247.1 found, 247.1 required. |
| Ex. 1-17 | 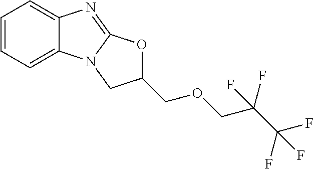 | 2-[(pentafluorethoxy) methyl]-2,3-dihydro [1,3]oxazolo[3,2-a] benzimidazole | LRMS m/z (M + H) 322.9 found, 323.2 required. |

The compounds in table 4 were synthesized without the addition of base in the 2-chlorobenzimidazole alkylation step.

TABLE 4

| | | | |
|---|---|---|---|
| Ex. 1-18 | 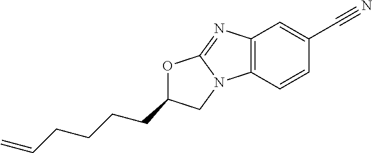 | (2R)-2-hex-5-en-1-yl- 2,3-dihydro[1,3] oxazolo[3,2-a] benzimidazole-7- carbonitrile | LRMS m/z (M + H) 268.0 found, 268.3 required |
| Ex. 1-19 | 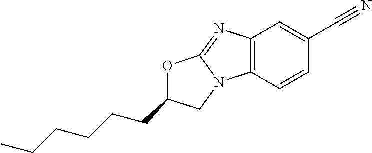 | (2R)-2-hexyl-2,3- dihydro[1,3]oxazolo [3,2-a]benzimidazole- 7-carbonitrile | LRMS m/z (M + H) 270.1 found, 270.3 required. |

The following compounds were synthesized according to Scheme 1 a followed by further manipulation.

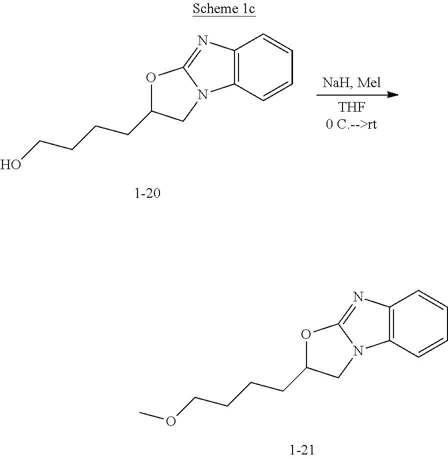

### Example 1-21

### 2-(4-methoxybutyl)-2,3-dihydrobenzo[d]oxazolo[3,2-a]imidazole (1-21)

Dry sodium hydride (4.7 mg, 0.19 mmol, 1.05 eq) was added to 4-(2,3-dihydrobenzo[d]oxazolo-[3,2-a]imidazol-2-yl)butan-1-ol (1-20) (41 mg, 0.18 mmol, 1 eq) in THF (1.2 mL) and the mixture was stirred at rt for 10 min. Methyl iodide (16.5 μL, 0.27 mmol, 1.5 eq) was added and the mixture stirred until disappearance of starting material as monitored by LC-MS. Water was added and the reaction mixture was extracted with ethyl acetate, dried with MgSO₄ followed by removal of the solvent under reduced pressure. The residue was purified by reverse phase liquid chromatography (Sunfire C18 OBD 5 μm, 20×150 mm column; 0-100% CH₃CN/H₂O gradient w/0.10% TFA present). The product in CH₃CN/H₂O was poured into aqueous sodium carbonate solution (2.0 M, 5 mL) and extracted with ethyl acetate. The organic layer was separated, dried over sodium sulfate and concentrated to yield 2-(4-methoxybutyl)-2,3-dihydrobenzo[d]oxazolo-[3,2-a]imidazole (1-21) as a white solid. ¹H NMR (500 MHz, CDCl₃): δ 7.53 (d, J=7.8 Hz, 1H); 7.19-7.10 (m, 3H); 5.41-5.34 (m, 1H); 4.34 (t, J=8.4 Hz, 1H); 3.88 (dd, J=8.8, 7.1 Hz, 1H); 3.41 (t, J=5.7 Hz, 2H); 3.34 (s, 3H); 2.10-2.01 (m, 1H); 1.96-1.87 (m, 1H); 1.70-1.53 (m, 4H). LRMS m/z (M+H) 247.1 found, 247.1 required.

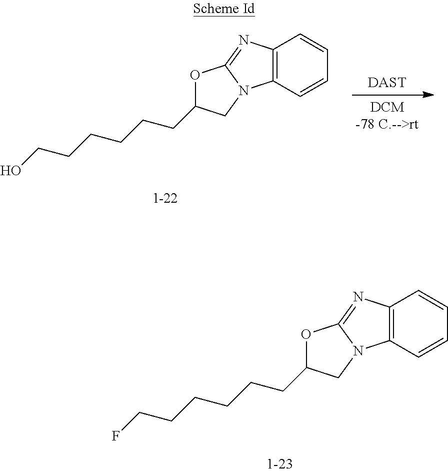

### Example 1-23

### 2-(6-fluorohexyl)-2,3-dihydrobenzo[d]oxazolo[3,2-a]imidazole (1-23)

To 6-(2,3-dihydrobenzo[d]oxazolo[3,2-a]imidazol-2-yl)hexan-1-ol (1-22) (78 mg, 0.3 mmol, 1 eq) in DCM was added DAST (0.20 mL, 1.5 mmol, 5 eq) at −78° C. and the mixture was stirred allowing the temperature to rise to room temperature. After overnight, starting material had disappeared (LC-MS analysis). Aqueous sodium carbonate was then added and the reaction mixture was extracted with DCM, dried with MgSO₄ and the solvent was removed under reduced pressure. The residue was purified by reverse phase liquid chromatography (Sunfire C18 OBD 5 μm, 20×150 mm column; 0-100% CH₃CN/H₂O gradient w/0.1% TFA). The product in CH₃CN/H₂O was poured into aqueous sodium carbonate solution (2.0 M, 5 mL) and extracted with ethyl acetate. The organic layer was separated, dried over sodium sulfate and concentrated to yield 2-(6-fluorohexyl)-2,3-dihydrobenzo[d]oxazolo[3,2-a]imidazole (1-23). ¹H NMR (400 MHz, CDCl₃): δ 7.54 (d, J=7.7 Hz, 1H); 7.20-7.10 (m, 3H); 5.43-5.33 (m, 1H); 4.51 (t, J=6.0 Hz, 1H); 4.43-4.30 (m, 2H); 3.87 (dd, J=8.9, 7.1 Hz, 1H); 2.12-1.96 (m, 1H); 1.95-1.83 (m, 1H); 1.77-1.42 (m, 8H). HRMS m/z (M+H) 263.1562 found, 263.1554 required.

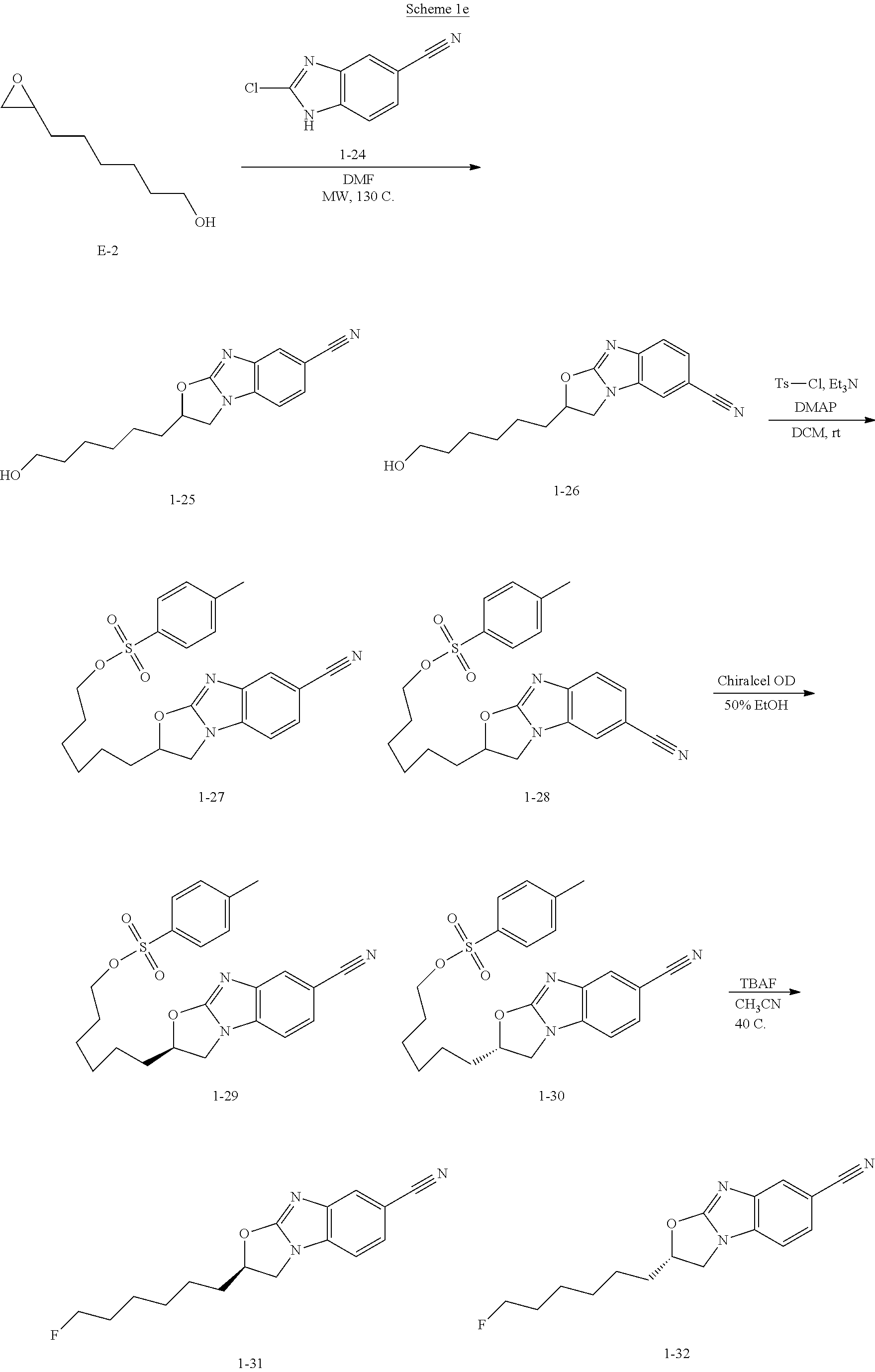

### 2-(6-hydroxyhexyl)-2,3-dihydrobenzo[d]oxazolo[3,2-a]imidazole-7-carbonitrile (1-25)

A mixture of 2-chloro-1H-benzo[d]imidazole-5-carbonitrile (1-24) (1.0 g, 5.6 mmol, 1 eq) and 6-(oxiran-2-yl)hexan-1-ol (E-2) (1.4 g, 8.5 mmol, 1.5 eq) in DMF (4 mL) was heated in a microwave at 130° C. until disappearance of starting material as monitored by LC-MS. Water was then added and the reaction mixture was extracted with ethyl acetate, dried with MgSO₄ and the solvent was removed under reduced pressure. The residue was purified by reverse phase liquid chromatography (Sunfire C18 OBD 5 μm, 20×150 mm column; 0-100% CH₃CN/H₂O gradient w/0.1% TFA). The product in CH₃CN/H₂O was poured into aqueous sodium carbonate solution (2.0 M, 5 mL) and extracted with ethyl acetate. The organic layer was separated, dried over sodium sulfate and concentrated to yield 2-(6-hydroxyhexyl)-2,3-dihydrobenzo[d]oxazolo-[3,2-a]imidazole-7-carbonitrile (1-25) and 2-(6-hydroxyhexyl)-2,3-dihydrobenzo[d]oxazolo[3,2-a]imidazole-6-carbonitrile (1-26) as a 1:1 regioisomeric mixture. ¹H NMR (1-25) (500 MHz, CDCl₃): δ 7.80 (s, 1H); 7.45 (m, 1H); 7.17 (d, J=8.2 Hz, 1H); 5.48-5.41 (m, 1H); 4.41 (t, J=8.6 Hz, 1H); 3.93 (t, J=8.1 Hz, 1H); 3.66 (t, J=6.5 Hz, 2H); 2.11-2.01 (m, 1H); 1.96-1.88 (m, 1H); 1.63-1.44 (m, 8H). LRMS m/z (M+H) 286.0 found, 286.1 required. ¹H NMR (1-26) (500 MHz, CDCl₃): δ 7.56 (d, J=8.3 Hz, 1H); 7.42 (m, 2H); 5.48-5.41 (m, 1H); 4.41 (t, J=8.6 Hz, 1H); 3.93 (t, J=8.1 Hz, 1H); 3.66 (t, J=6.5 Hz, 2H); 2.11-2.01 (m, 1H); 1.96-1.88 (m, 1H); 1.63-1.44 (m, 8H). LRMS m/z (M+H) 286.0 found, 286.1 required.

### (R)-6-(7-cyano-2,3-dihydrobenzo[d]oxazolo[3,2-a]imidazol-2-yl)hexyl 4-methylbenzene-sulfonate (1-29)

A mixture of 2-(6-hydroxyhexyl)-2,3-dihydrobenzo[d]oxazolo[3,2-a]imidazole-7-carbonitrile (1-25) and 2-(6-hydroxyhexyl)-2,3-dihydrobenzo[d]oxazolo[3,2-a]imidazole-6-carbonitrile (1-26) (780 mg, 2.7 mmol, 1 eq) was dissolved in DCM (5.5 mL) and triethylamine (1.1 mL, 8.2 mmol, 3 eq), tosyl chloride (573 mg, 3.0 mmol, 1.1 eq) and DMAP (33 mg, 0.3 mmol, 0.1 eq) were added. The mixture was stirred overnight at ambient temperature, after which LC-MS analysis showed complete consumption of the starting material, then quenched with water. The aqueous phase was extracted with DCM and the combined organic extracts were washed with brine and dried with MgSO₄. The resulting crude was purified via flash chromatography on a 80 g silica gel column (gradient elution 0 to 100% ethyl acetate in hexanes) to yield 6-(7-cyano-2,3-dihydrobenzo[d]oxazolo[3,2-a]imidazol-2-yl)hexyl 4-methylbenzenesulfonate (1-27) and 6-(6-cyano-2,3-dihydrobenzo[d]oxazolo[3,2-a]imidazol-2-yl)hexyl 4-methylbenzenesulfonate (1-28), both as a white solid. Chiral separation on Chiralcel OD column (gradient elution 50% EtOH in hexanes with 0.1% DEA) afforded (R)-6-(7-cyano-2,3-dihydrobenzo[d]oxazolo[3,2-a]imidazol-2-yl)hexyl 4-methylbenzenesulfonate (1-29) as a white solid. ¹H NMR (1-27 and 1-29) (500 MHz, CDCl₃): δ 7.80-7.75 (m, 3H); 7.40 (dd, J=8.2, 1.5 Hz, 1H); 7.35 (d, J=8.0 Hz, 2H); 7.19-7.10 (m, 1H); 5.43 (qd, J=7.6, 5.2 Hz, 1H); 4.45-4.37 (m, 1H); 4.03 (t, J=6.3 Hz, 2 H); 3.93 (dd, J=9.2, 7.2 Hz, 1H); 2.45 (s, 3H); 2.06-1.96 (m, 1H); 1.93-1.84 (m, 1H); 1.69-1.44 (m, 4H); 1.41-1.36 (m, 4H). LRMS m/z (M+H) 440.1 found, 440.1 required. ¹H NMR (1-28) (500 MHz, CDCl₃): δ 7.79 (d, J=8.0 Hz, 2H); 7.54 (t, J=8.2 Hz, 1H); 7.48-7.41 (m, 2H); 7.35 (d, J=8.0 Hz, 2H); 5.50-5.42 (m, 1H); 4.42 (t, J=8.5 Hz, 1H); 4.04 (t, J=6.3 Hz, 2H); 3.93 (dd, J=9.0, 7.2 Hz, 1H); 2.45 (s, 3H); 2.07-1.97 (m, 1H); 1.95-1.85 (m, 1H); 1.69-1.44 (m, 4H); 1.41-1.38 (m, 4H). LRMS m/z (M+H) 440.1 found, 440.1 required.

### Example 1-31

### (R)-2-(6-fluorohexyl)-2,3-dihydrobenzo oxazolo[3,2-a]imidazole-7-carbonitrile (1-31)

(R)-6-(7-cyano-2,3-dihydrobenzo[d]oxazolo[3,2-a]imidazol-2-yl)hexyl 4-methylbenzenesulfonate (1-29) (30 mg, 0.07 mmol, 1 eq) was dissolved in CH₃CN (1.5 mL) and TBAF (70 μL, 0.07 mmol, 1.02 eq) was added. The mixture was stirred at 40° C. until disappearance of the starting material as monitored by LC-MS, following purification by reverse phase liquid chromatography (Sunfire C18 OBD 5 μm, 20×150 mm column; 0-100% CH₃CN/H₂O gradient w/0.1% TFA). The product in CH₃CN/H₂O was poured into aqueous sodium carbonate solution (2.0 M, 5 mL) and extracted with ethyl acetate. The organic layer was separated, dried over sodium sulfate and concentrated to yield R)-2-(6-fluorohexyl)-2,3-dihydrobenzo[d]oxazolo[3,2-a]imidazole-7-carbonitrile (1-31) as a white solid. ¹H NMR (500 MHz, CDCl₃): δ 7.81 (s, 1H); 7.42 (d, J=8.2 Hz, 1H); 7.18 (d, J=8.0 Hz, 1H); 5.49-5.43 (m, 1H); 4.50 (t, J=6.0 Hz, 1H); 4.44-4.38 (m, 2H); 3.94 (dd, J=9.1, 7.2 Hz, 1H); 2.09-2.02 (m, 1H); 1.96-1.88 (m, 1H); 1.74 (s, 1H); 1.71-1.64 (m, 1H); 1.63 (s, 2H); 1.55 (s, 2 H); 1.26 (s, 2H). LRMS m/z (M+H) 288.1 found, 288.1 required.

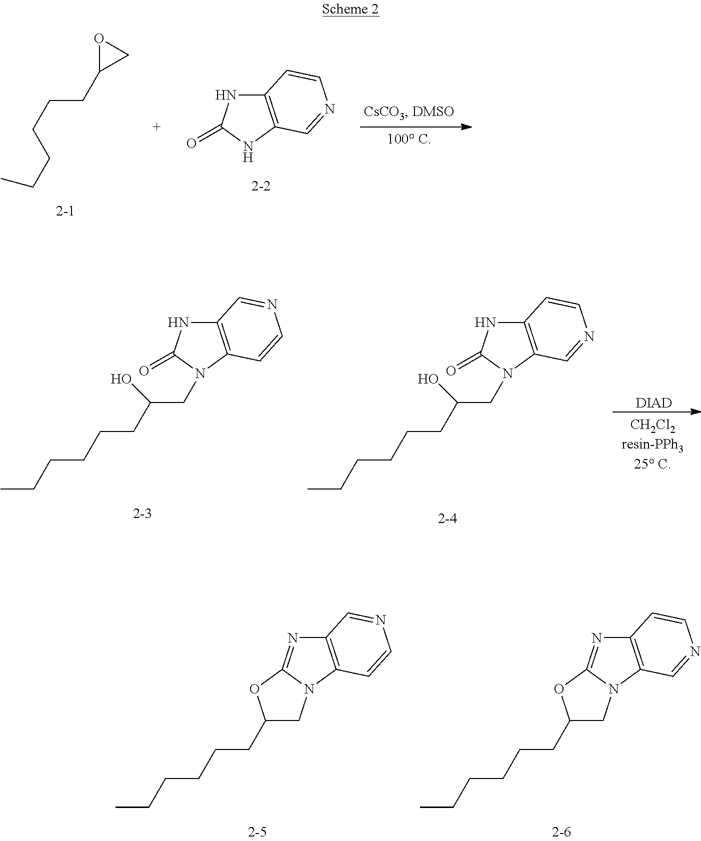

### 1-(2-hydroxyoctyl)-1,3-dihydro-2H-imidazo[4,5-c]pyridin-2-one (2-3) and 3-(2-hydroxyoctyl)-1,3-dihydro-2H-imidazo[4,5-c]pyridin-2-one (2-4)

A mixture of 2-hexyloxirane (2-1) (195 mg, 1.525 mmol, 1.03 equiv), 1,3-dihydro-2H-imidazo[4,5-c]pyridin-2-one (2-2) (200 mg, 1.480 mmol, 1.0 equiv) and cesium carbonate (241 mg, 0.740 mmol, 0.5 equiv) in DMF (3 mL) was heated at 130 C in a microwave until disappearance of SM as monitored by LC-MS. The mixture was cooled, the crude was filtered through a silica plug and the filtrate was purified by reverse phase liquid chromatography (Sunfire C18 OBD 5 um, 20×150 mm column; 0-100% CH₃CN/H₂O gradient w/0.10% TFA present). The product in CH₃CN/H₂O was poured into aqueous sodium carbonate solution (2.0 M, 5 mL) and extracted with dichloromethane. The organic layer was separated, dried over sodium sulfate and concentrated to yield pure 1-(2-hydroxyoctyl)-1,3-dihydro-2H-imidazo[4,5-c]pyridin-2-one (2-3) and 3-(2-hydroxyoctyl)-1,3-dihydro-2H-imidazo[4,5-c]pyridin-2-one (2-4) as a white solid.

### Examples 2-5 and 2-6

### 2-hexyl-2,3-dihydro[1,3]oxazolo[3′,2′:1,2]imidazo[4,5-c]pyridine (2-5) and 2-hexyl-2,3-dihydro[1,3]oxazolo[2′,3:2,3]imidazo[4,5-c]pyridine (2-6)

A mixture of 1-(2-hydroxyoctyl)-1,3-dihydro-2H-imidazo[4,5-c]pyridin-2-one (2-3) and 3-(2-hydroxyoctyl)-1,3-dihydro-2H-imidazo[4,5-c]pyridin-2-one (2-4) (110 mg, 0.418 mmol, 1.00 equiv), PS-triphenylphosphine (685 mg, 1.253 mmol, 3 equiv, 1.83 mmol/g) and DIAD (0.122 mL, 0.627 mmol, 1.5 equiv) in DCM (5 mL) was rotated at RT until complete disappearance of SM as monitored by LC-MS. The resin was removed by filtration, the solvent was removed and the residue was purified by reverse phase liquid chromatography (Sunfire C18 OBD 5 μm, 20×150 mm column; 0-100% CH₃CN/H₂O gradient w/0.10% TFA present). The product in CH₃CN/H₂O was poured into aqueous sodium carbonate solution (2.0 M, 5 mL) and extracted with dichloromethane. The organic layer was separated, dried over sodium sulfate and concentrated to yield 2-hexyl-2,3-dihydro[1,3]oxazolo[3′,2′:1,2]imidazo[4,5-c]pyridine (2-5) and 2-hexyl-2,3-dihydro[1,3]oxazolo[2′,3′:2,3]imidazo[4,5-c]pyridine (2-6) as a 2:1 regioisomeric mixture. ¹H NMR (2-5) (500 MHz, CDCl₃): δ 8.78 (s, 1H); 8.33 (d, J=5.3 Hz, 1H); 7.09 (d, J=5.4 Hz, 1H); 5.44 (dd, J=7.8, 5.8 Hz, 1H); 4.99-4.94 (m, 1H); 4.38 (t, J=8.5 Hz, 1H); 3.92 (dd, J=9.1, 7.2 Hz, 1H); 2.07-2.03 (m, 1H); 1.91-1.88 (m, 1H); 1.52-1.45 (m, 2H); 1.40 (t, J=7.5 Hz, 2H); 1.33-1.30 (m, 2H); 1.26 (d, J=6.3 Hz, 2H); 0.90 (t, 6.7 Hz, 2H). LRMS m/z (M+H) 246.1 found, 246.3 required.

¹H NMR (2-6) (500 MHz, CDCl₃): δ 8.48 (s, 1H); 8.37 (d, J=5.4 Hz, 1H); 7.44 (d, J=5.5 Hz, 1H); 5.44 (dd, J=7.8, 5.8 Hz, 1H); 4.99-4.94 (m, 1H); 4.43 (t, J=8.5 Hz, 1H); 3.96 (dd, J=9.0, 7.3 Hz, 1H); 2.07-2.03 (m, 1H); 1.91-1.88 (m, 1H); 1.52-1.45 (m, 2H); 1.40 (t, J=7.5 Hz, 2H); 1.33-1.30 (m, 2H); 1.26 (d, J=6.3 Hz, 2H); 0.90 (t, J=6.7 Hz, 2H). LRMS m/z (M+H) 246.1 found, 246.3 required.

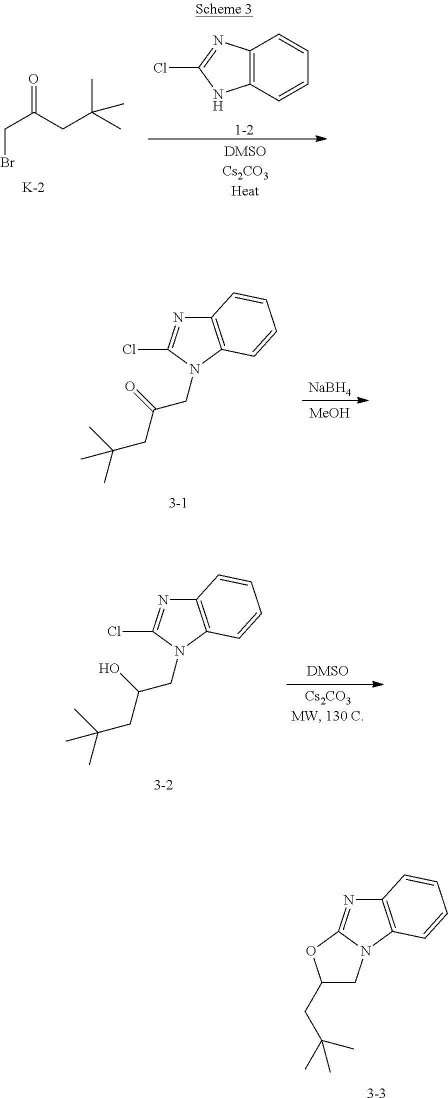

### 1-(2-chloro-1H-benzimidazol-1-yl)-4,4-dimethylpentan-2-one (3-1)

2-Chlorobenzimidazole (1-2) (198 mg, 1.3 mmol, 1 eq.), 1-bromo-4,4-dimethylpentan-2-one (251 mg, 1.3 mmol, 1 eq.) and cesium carbonate (635 mg, 1.3 mmol, leg.) were mixed in DMSO (2 mL) and the resulting suspension was heated at 80 C until disappearance of the starting material as monitored by LC-MS. Water was added and the 1-(2-chloro-1H-benzimidazol-1-yl)-4,4-dimethylpentan-2-one (3-1) precipitate was filtered and washed with water. No further purification was needed.

### 1-(2-chloro-1H-benzimidazol-1-yl)-4,4-dimethylpentan-2-ol (3-2)

Sodium borohydride (91 mg, 2.4 mmol, 3 eq) was added to a solution of 1-(2-chloro-1H-benzimidazol-1-yl)-4,4-dimethylpentan-2-one (3-1) (213 mg, 0.8 mmol, 1 eq) in MeOH (2 mL) at rt, and the solution was stirred until disappearance of starting material as monitored by LC-MS. Aqueous ammonium chloride was added and the reaction mixture was extracted with CHCl₃, dried with MgSO₄ and the solvent was removed under reduced pressure. The residue was purified by reverse phase liquid chromatography (Surefire C18 OBD 5 μm, 20×150 mm column; 0-100% CH₃CN/H₂O gradient w/0.10% TFA present). The product in CH₃CN/H₂O was poured into aqueous sodium carbonate solution (2.0 M, 5 mL) and extracted with dichloromethane. The organic layer was separated, dried over sodium sulfate and concentrated to yield 1-(2-chloro-1H-benzimidazol-1-yl)-4,4-dimethylpentan-2-ol (3-2) as a white solid.

### Example 3-3

### 2-(2,2-dimethylpropyl)-2,3-dihydro[1,3]oxazolo[3,2-a]benzimidazole (3-3)

A mixture of 1-(2-chloro-1H-benzimidazol-1-yl)-4,4-dimethylpentan-2-ol (3-2) (265 mg, 1 mmol, 1 eq.), and cesium carbonate (325 mg, 1 mmol, 1 eq.) in DMF (2 mL) was heated at 130 C in a microwave until disappearance of SM as monitored by LC-MS. The reaction was cooled and filtered. The filtrate was purified by reverse phase liquid chromatography (Sunfire C18 OBD 5 μM, 20×150 mm column; 0-100% CH₃CN/H₂O gradient w/0.10% TFA present). The product in CH₃CN/H₂O was poured into aqueous sodium carbonate solution (2.0 M, 5 mL) and extracted with dichloromethane. The organic layer was separated, dried over sodium sulfate and concentrated to yield (2S)-2-{[4-(1-methyl-1H-pyrazol-5-yl)phenoxy]methyl}-2,3-dihydro[1,3]oxazolo[3,2-c]benzimidazole (3-3) as a white solid. ¹H NMR (400 MHz, CDCl₃) δ 7.52 (dd, 1H, J=7.7 Hz), 7.20-7.08 (m, 3H), 5.49-5.43 (m, 1H); 4.37 (dd, 1H, J=8.3, 8.2 Hz); 3.82 (dd, 1H, J=11.3, 8.4 Hz), 2.07 (dd, 1H, J=8.4, 6.2 Hz), 1.70 (dd, 1H, J=14.7, 4.0 Hz), 1.07 (s, 9H). LRMS m/z (M+H) 231.1 found, 231.1 required.

The compounds shown in Table 5 were synthesizes according to Scheme 3.

TABLE 5

| | | | |
|---|---|---|---|
| Ex. 3-4 | 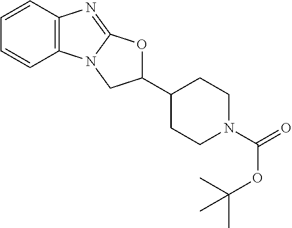 | Tert-butyl 4-92,3- dihydro[1,3]oxazolo [3,2-a]benzimidazole- 2-yl)piperidine-l- carboxylate | LRMS m/z (M + H) 344.1 found, 344.4 required. |
| Ex. 3-5 | 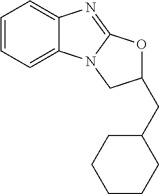 | 2-(cyclohexyl- methyl)-2,3- dihydro[1,3]oxazolo [3,2-a]benzimidazole | LRMS m/z (M + H) 257.1 found, 257.3 required. |
| Ex. 3-6 | 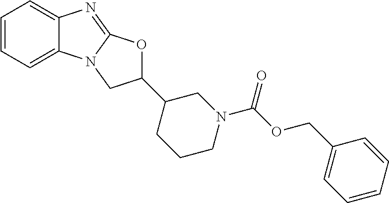 | Benzyl 3-(2,3- dihydro[1,3]oxazolo [3,2-a]benzimidazole- 2-yl)piperidine-1- carboxylate | LRMS m/z (M + H) 378.1 found, 377.4 required. |
| Ex. 3-7 | 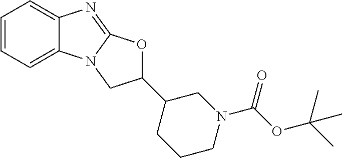 | tert-butyl 3-(2,3- dihydro[1,3]oxazolo [3,2-a]benzimidazole- 2-yl)piperidine-1- carboxylate | LRMS m/z (M + H) 344.1 found, 343.4 required. |

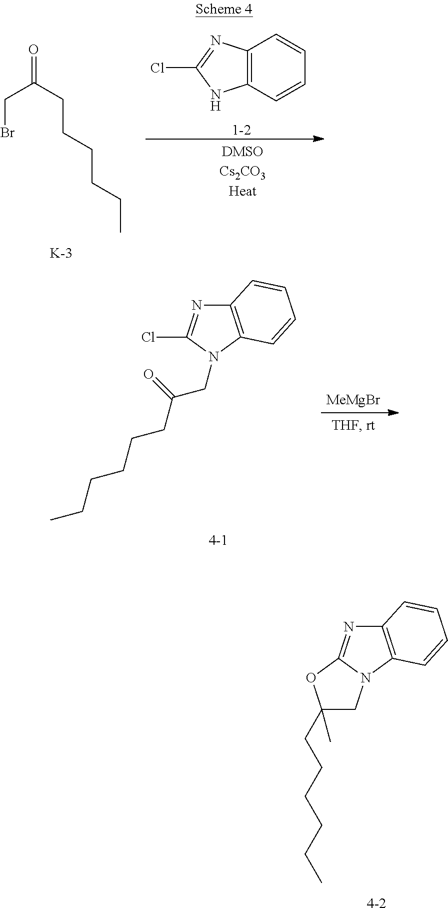

### 1-(2-chloro-1H-benzimidazol-1-yl)octan-2-one (4-1)

2-Chlorobenzimidazole (1-2) (1.1 g, 7.2 mmol, 1 eq), cesium carbonate (2.8 g, 8.53 mmol, 1.2 eq) and 1-bromooctan-2-one (K-3) (1.5 g, 7.2 mmol, 1 eq) were mixed in DMSO (9 mL) and stirred at room temperature until complete consumption of the starting materials as monitored by LC-MS. Water was added and the sticky precipitate was filtered and further purified by silica gel chromatography (EtOAc/hexane gradient) to yield 1-(2-chloro-1H-benzimidazol-1-yl)octan-2-one (4-1) as a white flaky solid.

### Example 4-2

### 2-hexyl-2-methyl-2,3-dihydro[1,3]oxazolo[3,2-a]benzimidazole (4-2)

1-(2-Chloro-1H-benzimidazol-1-yl)octan-2-one (4-1) (30 mg, 0.11 mmol, 1 eq.) was dissolved in THF and methylmagnesium bromide (1.4 M solution in Tol/THF 75:25; 300 μL, 3.8 eq.) was added at room temperature. The mixture was stirred until consumption of the starting material, quenched with saturated NH₄Cl, the aqueous phase was extracted with CHCl₃ and the combined organic extracts were washed with brine and dried with MgSO₄. The resulting crude was purified by reverse phase liquid chromatography (Sunfire C18 OBD 5 μm, 20×150 mm column; 0-100% CH₃CN/H₂O gradient w/0.10% TFA present). The product in CH₃CN/H₂O was poured into aqueous sodium carbonate solution (2.0 M, 5 mL) and extracted with dichloromethane. The organic layer was separated, dried over sodium sulfate and concentrated to yield 2-hexyl-2-methyl-2,3-dihydro[1,3]oxazolo[3,2-a]benzimidazole (4-2) as a white solid. ¹H NMR (400 MHz, CDCl₃) δ 7.52 (d, 1H, J=7.7 Hz), 7.07-7.10 (m, 3H), 4.15 (dd, 2H, J=6.6, 6.6 Hz); 1.9-2.1 (m, 2H), 1.8-1.9 (m, 2H), 1.5-1.75 (m, 5H), 1.23-1.45 (m, 4H), 0.9 (bt, 3H, J=6.7 Hz). LRMS m/z (M+H) 259.1 found, 259.2 required.

The compounds shown in Table 6 were synthesizes according to Scheme 4.

TABLE 6

| | | | |
|---|---|---|---|
| Ex. 4-3 | 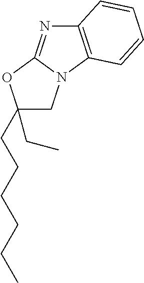 | 2-hexyl-2-methyl-2,3- dihydro[1,3]oxazolo [3,2-a]benzimidazole | LRMS m/z (M + H) 273.0 found, 273.2 required. |
| Ex. 4-4 | 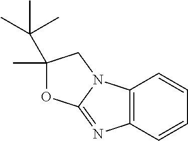 | 2-tert-butyl-2-methyl- 2,3-dihydro[1,3]- oxazolo[3,2-a] benzimidazole | LRMS m/z (M + H) 231.1 found, 231.2 required. |

What is claimed is:

1. A compound according to Formula I 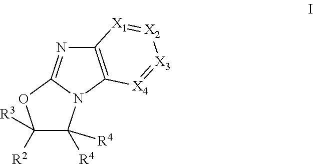
or a pharmaceutically acceptable salt thereof, wherein:
X₁, X₂, X₃ and X₄ are independently selected from the group consisting of C(R¹) and N,
wherein each R¹ is independently selected from the group consisting of:
(1) H,
(2) halo,
(3) C₁₋₈alkyl,
(4) C₂₋₆alkenyl,
(5) C₂₋₆alkynyl,
(6) C₃₋₆cycloalkyl,
(7) C₁₋₆alkoxy,
(8) C₃₋₆cycloalkoxy,
(9) —CN,
(10) —OH,
(11) —C(O)—O—C₁₋₄alkyl,
(12) —C(O)—C₁₋₄alkyl,
(13) —N(R)₂,
(14) —C(O)—N(R)₂,
(15) —S(O)ₖ—C₁₋₄alkyl, wherein k is 0, 1 or 2,
(16) -aryl,
(17) -heteroaryl, optionally substituted with 1 to 2 methyl groups,
(18) —C(O)-aryl,
(19) —N(R)-aryl,
(20) benzyl,
(21) benzyloxy,
(22) —CO₂H,
(23) —SH,
(24) —SO₂N(R)R,
(25) —N(R)C(O)N(R)R,
(26) —N(R)C(O)C₁₋₄alkyl,
(27) —N(R)SO₂N(R)R,
(28) trimethylsilyl and
(29) 1-methylsiletan-1-yl,
wherein groups (3) through (8) above are optionally substituted from one up to the maximum number of substitutable positions with one or more substituents independently selected from the group consisting of: OH, CN, oxo, halo, C₁₋₄alkoxy and C₁₋₄alkylamino,
and two R¹ substituents on adjacent atoms may be joined together with the atoms to which they are attached to form a 5- or 6-membered saturated or partially unsaturated monocyclic ring optionally containing 1 or 2 heteroatoms selected from O, S and N, said ring optionally substituted with oxo or 1 to 3 halo groups, or both, and said ring optionally fused with a benzo group;
R² is selected from the group consisting of:
(1) 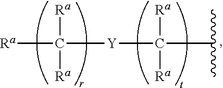
wherein Y is O or a bond, r and t are independently 0 to 9, except that r+t is greater than 4, and each R^{a} is independently selected from H, halo and C₁₋₄alkyl, optionally substituted with 1 to 3 halo groups, and two R^{a} groups on adjacent carbon atoms may be joined together to form a double bond,
(2) C₃₋₁₀cycloalkyl or C₃₋₁₀cycloalkyl —(CH₂)_{q}—, wherein q is 1 to 4,
(3) CF₃,
(4) text-butyl,
(5) 2,2-dimethylpropyl and
(6) 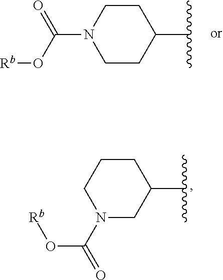
wherein R^{b} is selected from C₁₋₆alkyl, phenyl and benzyl, any of which may be optionally substituted with 1 to 3 halo groups;
R³ and each R⁴ are independently selected from the group consisting of: H, halo and C₁₋₄alkyl, said C₁₋₄alkyl optionally substituted with oxo and 1 to 3 substituents independently selected from the group consisting of: F, OH and N(R)₂; and
each R is independently selected from the group consisting of: H and C₁₋₄alkyl.

2. The compound according to claim 1 wherein R³ is methyl or ethyl.

3. The compound according to claim 1 wherein R² is

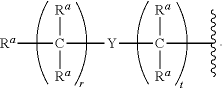

4. The compound according to claim 1 wherein R² is C₃₋₁₀cycloalkyl or C₃₋₁₀cycloalkyl —(CH₂)_{q}—, wherein q is 1 to 4.

5. The compound according to claim 1 wherein R² is

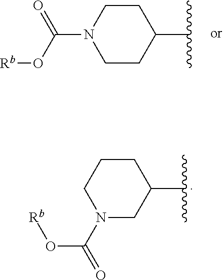

6. The compound according to claim 1 of Formula Ia 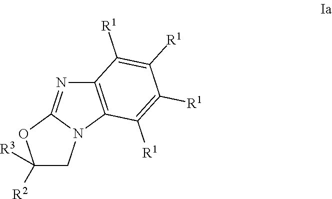
or a pharmaceutically acceptable salt thereof.

7. The compound according to claim 6 wherein R² is

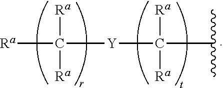

8. The compound according to claim 1 of Formula Ib 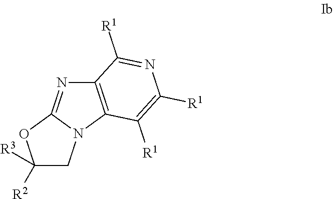
or a pharmaceutically acceptable salt thereof.

9. The compound according to claim 8 wherein R² is

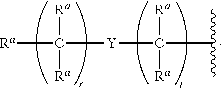

10. A compound selected from the following group: 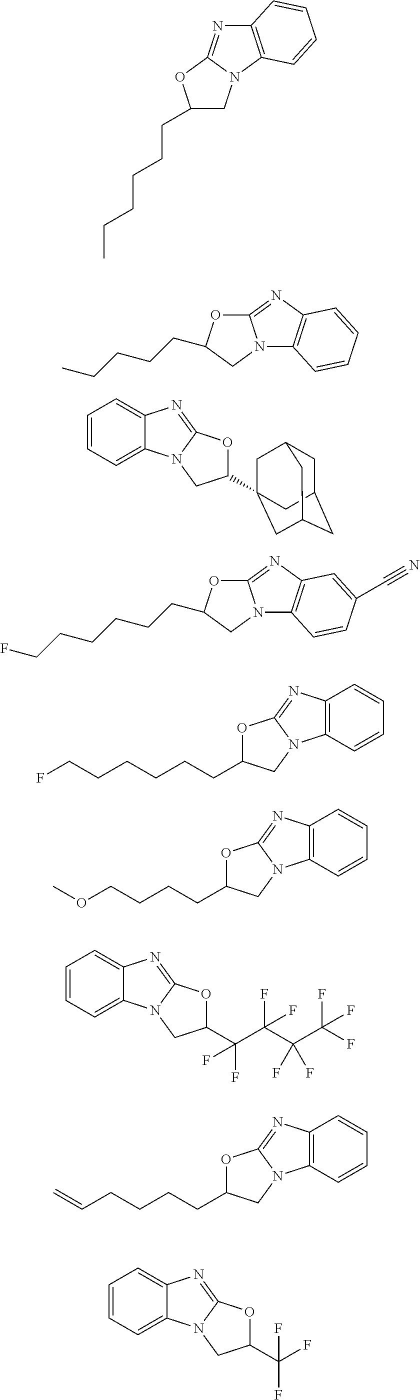 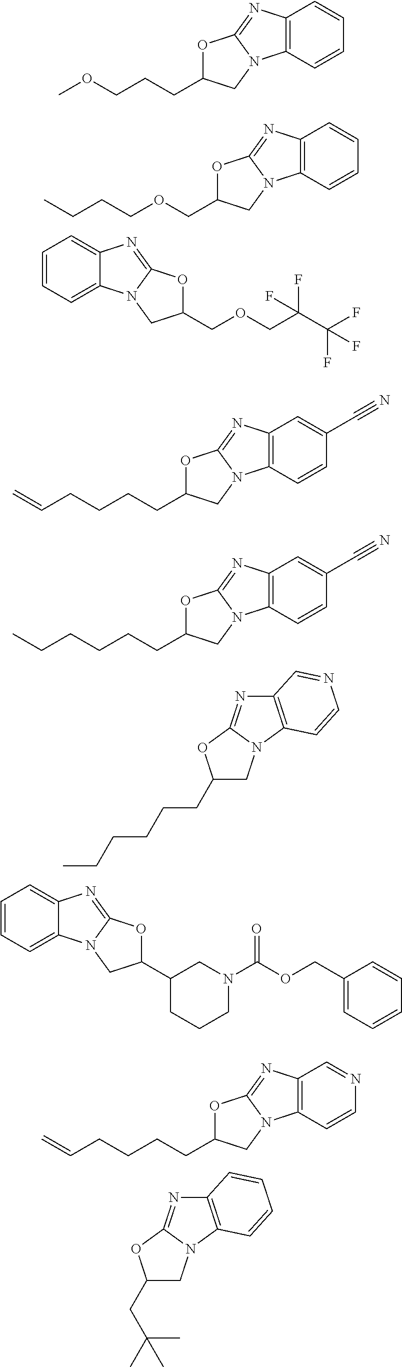 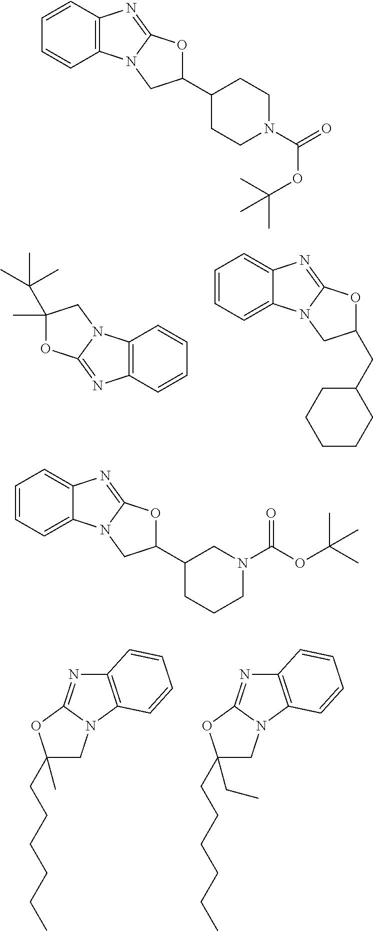
and pharmaceutically acceptable salts of the foregoing compounds.

11. A pharmaceutical composition comprising a compound according to claim 1 in combination with a pharmaceutically acceptable carrier.

12. A method for treating a neurological or psychiatric disorder associated with glutamate dysfunction in a patient in need thereof comprising administering to the patient a therapeutically effective amount of a compound according to claim 1.

13. The method according to claim 12 wherein the neurological or psychiatric disorder associated with glutamate dysfunction is schizophrenia.
